(19) United States
(12) Patent Application Publication
(10) Pub. No.: US 2015/0152447 A1
(43) Pub. Date: Jun. 4, 2015

(54) YEAST CELL WITH INACTIVATED GLYCEROL-3-PHOSPHATE DEHYDROGENASE AND ACTIVATED GLYCERALDEHYDE-3-PHOSPHATE DEHYDROGENASE AND METHOD OF PRODUCING LACTATE USING THE SAME

(71) Applicant: Samsung Electronics Co., Ltd., Suwon-si (KR)

(72) Inventors: Sungsoo Kim, Hwaseong-si (KR); Jiyoon Song, Seoul (KR); Changduk Kang, Gwacheon-si (KR); Juyoung Lee, Daegu (KR); Seunghyun Lee, Asan-si (KR); Kwangmyung Cho, Seongnam-si (KR)

(21) Appl. No.: 14/559,426

(22) Filed: Dec. 3, 2014

(30) Foreign Application Priority Data
Dec. 3, 2013 (KR) 10-2013-0149492

(51) Int. Cl. C12P 7/56; C12N 9/04; C12N 15/81; C12N 9/02

(52) U.S. Cl. CPC C12P 7/56; C12N 9/0008; C12N 9/0006; C12N 15/81; C12Y 102/01012; C12Y 101/01027

(57) ABSTRACT

A genetically modified yeast cell comprising increased glyceraldehyde-3-phosphate dehydrogenase activity converting glyceraldehyde-3-phosphate to 1,3-diphosphoglycerate as compared to a parent yeast cell of the same type, and reduced glycerol-3-phosphate dehydrogenase activity converting dihydroxyacetone phosphate to glycerol-3-phosphate compared to a parent yeast cell of the same type, and related compositions and methods.

### CROSS-REFERENCE TO RELATED APPLICATIONS

This application claims the benefit of Korean Patent Application No. 10-2013-0149492, filed on Dec. 3, 2013, in the Korean Intellectual Property Office, the entire disclosure of which is hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: One 36,419 bytes ASCII (Text) file named “716771_ST25.TXT,” created Dec. 2, 2014.

### BACKGROUND

1. Field

The present disclosure relates to a yeast cell with an inactivated or depressed glycerol-3-phosphate dehydrogenase and a method of producing lactate using the yeast cell.

2. Description of the Related Art

Lactate is an organic acid that is broadly used in various industrial fields, such as food, pharmaceutics, chemicals, and electronics. Lactate is colorless, odorless, and a low-volatile material that dissolves well in water. Lactate is non-toxic to the human body and thus may be used as a flavor agent, a taste agent, or a preserving agent. Also, lactate is an environment-friendly alternative polymer material and a raw material of a polylactic acid (PLA) that is biodegradable plastic.

PLA is a polyester-based resin that is ring-open polymerized by converting it into lactide, which is a dimer, for technical polymerization and may be variously processed into a film, sheet, fiber, plastic, etc. Thus, demands for PLA as bioplastic have recently increased to broadly replace conventional typical petrochemical plastic, such as polyethylene (PE), polypropylene (PP), polyethylene terephthalate (PET), or polystylene (PS).

In addition, lactate includes both a hydroxyl group and a carboxyl group at and thus is highly reactive. Accordingly, lactate is easily converted into an industrially important compound, such as lactate ester, acetaldehyde, or propyleneglycol, and thus has received attention as an alternative chemical material of the next generation in chemical industry.

Currently, lactate is produced by an industrially petrochemical synthesis process and a biotechnological fermentation process. The petrochemical synthesis process is performed by oxidizing ethylene derived from crude oil, preparing lactonitrile through addition of hydrogen cyanide after acetaldehyde, purifying by distillation, and hydrolyzing by using chloric acid or phosphoric acid. Also, the biotechnological fermentation process is used to manufacture lactate from a reproducible carbon hydrate, such as, starch, sucrose, maltose, glucose, fructose, or xylose, as a substrate.

Therefore, a strain for efficiently producing lactate and a lactate production method using the strain are needed.

### SUMMARY

Provided is a yeast cell with improved lactate productivity. In particular, provided is a genetically modified yeast cell comprising increased glyceraldehyde-3-phosphate dehydrogenase activity converting glyceraldehyde-3-phosphate to 1,3-diphosphoglycerate as compared to a parent yeast cell of the same type, and reduced glycerol-3-phosphate dehydrogenase activity converting dihydroxyacetone phosphate to glycerol-3-phosphate compared to a parent yeast cell of the same type. A method of preparing the yeast cell also is provided.

Also provided is a method of efficiently producing lactate by using the yeast cell.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings of which:

FIG. 1 illustrates a pathway of producing lactate from a yeast cell capable of producing lactate;

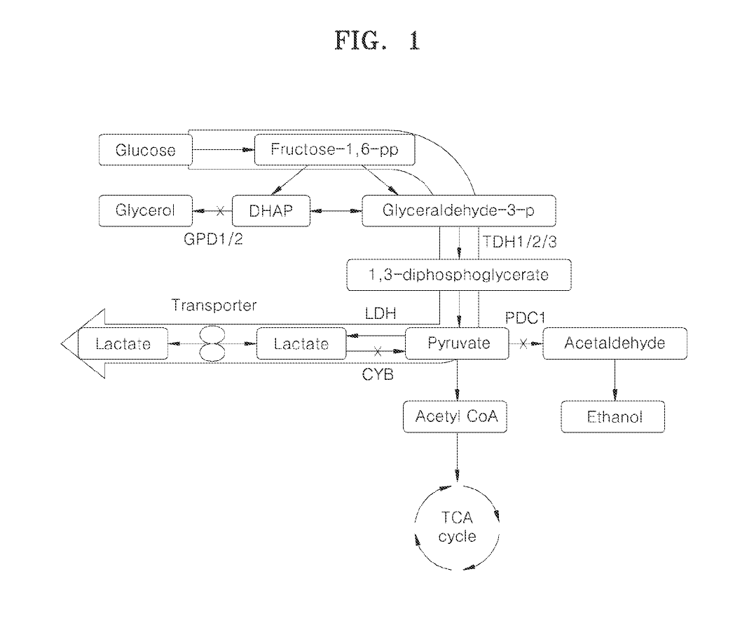

FIG. 2 is a schematic of an overexpression vector for overexpressing TDH1;

FIG. 3 is a schematic of a pUC19-HIS3 vector;

FIG. 4 is a schematic of a pUC19-PDCp-TDH1-HIS3 vector;

FIG. 5 illustrates a process of preparing a KCTC12415BP Δ GPD2+TDH1 strain via deletion of GPD2 from a mother strain KCTC12415BP; and

FIG. 6 is a graph illustrating the culturing characteristics of the mother strain KCTC12415BP;

FIG. 7 is a graph illustrating the culturing characteristics of the strain KCTC12415BP; and

FIG. 8 illustrates the culturing characteristics of the strain, c, KCTC12415BPΔGPD2+TDH1.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be constructed as being limited to the descriptions set forth herein. Accordingly, the embodiments are merely described below, by referring to the figures, to explain aspects of the present description. As used herein, the term “and/or” includes any and all combinations of one or more of the associated listed items.

According to an embodiment of the present invention, a genetically modified yeast cell is provided with a deletion or disruption mutation of a gene encoding a polypeptide that converts dihydroxyacetone phosphate to glycerol-3-phosphate and reduced (inactivated or depressed) glycerol-3-phosphate dehydrogenase activity converting dihydroxyacetone phosphate to glycerol-3-phosphate as compared to a parent yeast cell not having a deletion or disruption mutation of the gene encoding a polypeptide that converts dihydroxyacetone phosphate to glycerol-3-phosphate, and increased activity of converting glyceraldehyde-3-phosphate to 1,3-diphosphoglycerate as compared to a parent cell not having an increased glyceraldehyde-3-phosphate dehydrogenase activity.

As used herein, an “inactivated”, “reduced”, “depressed”, or “attenuated” activity of an enzyme, a polypeptide, or a cell, or having an activity that is “inactivated” or “reduced” or “depressed,” denotes a cell, an enzyme (e.g., isolated enzyme or enzyme in a cell), or a polypeptide having an activity that is lower than the same activity measured in a parent yeast cell of the comparably same type or the original enzyme or polypeptide. Reduced activity encompasses no activity. Activity may be reduced by any amount. For example, an enzyme conversion activity from a substrate to a product with respect to a corresponding enzyme may be about 20% or more, about 30% or more, about 40% or more, about 50% or more, about 55% or more, about 60% or more, about 70% or more, about 75% or more, about 80% or more, about 85% or more, about 90% or more, about 95% or more, or about 100% reduced than the biochemical conversion activity by an enzyme that is produced by a parent yeast cell of the same type. The cells having reduced activity of the enzyme may be confirmed by using methods commonly known in the art. The term “inactivation” may refer to generation of a gene that is rendered inexpressible or a gene that is expressible but produces a product having no activity. The term “reduction” or “depression”, or “attenuation” may refer to generation of a less expressible gene compared to the expressability of said gene in a non-manipulated yeast cell, for example, a genetically non-manipulated yeast cell, or a gene that is expressible but produces a product with lower activity than a non-manipulated yeast cell, for example, a genetically non-manipulated yeast cell. An activity of the enzyme may be reduced (e.g., inactivated) due to substitution, addition, or deletion of a part or all of a gene encoding the enzyme. For example, inactivation or reduction of the enzyme may be caused by homologous recombination or may be performed by transforming the cell with a vector including a part of sequence of the gene, culturing the cell so that the sequence may homogonously recombined with an endogenous gene of the cell, and then selecting cells, in which homologous recombination occurred, using a selection marker.

As used herein, the term “activity increase”, “enzyme activity increase”, “increased activity”, or “increased enzyme activity” denotes that a cell or enzyme (isolated or within a cell) has an increased activity level compared to an activity level of a comparable parent cell. Activity can be increased by any amount. For instance, an enzyme conversion activity from a substrate to a product with respect to a corresponding enzyme may be at least about 5%, at least about 10%, at least about 15%, at least about 20%, at least about 30%, at least about 50%, at least about 60%, at least about 70%, or at least about 100% increased compared to the same biochemical conversion activity of a parent cell or wild-type enzyme. A cell having an increased enzyme activity of an enzyme may be confirmed by using any method commonly known in the art.

The term “parent cell” denotes a cell not having a specific genetic modification resulting in a genetically engineered cell. The parent cell also denotes a cell which is not applied a genetic modification of interest gene for identifying biochemical and/or genetic function of the interest gene. The parent cell also may refer to an original cell, for example, a non-engineered cell of the same type as an engineered yeast cell. With respect to a particular genetic modification, the “parent cell” can be a cell that lacks the particular genetic modification, but is identical in all other respects. Thus, a parent cell can be a cell used as starting material to produce a genetically engineered yeast cell having an activated or increased activity of a given protein (e.g., a protein having a sequence identity of about 95% or more to a glyceraldehyde-3-phosphate dehydrogenase), or a genetically engineered yeast cell having an reduced activity of a given protein (e.g., a protein having a sequence identity of about 95% or more to a glycerol-3-phosphate dehydrogenase). The parent cell may be also referred to “mother cell”. The term “wild-type” enzyme, polypeptide or polynucleotide denotes an enzyme, a polypeptide or a polynucleotide not having a specific genetic modification resulting in a genetically engineered enzyme, polypeptide or polynucleotide.

The increased activity of the enzyme or polypeptide may occur due to an increased expression or an increased specific activity. The increased expression may occur by introducing a polynucleotide encoding a polypeptide into a cell repetitively, or mutating a regulatory region of the polynucleotide. A polynucleotide that is introduced may increase copy number of the polynucleotide in the cell. A polynucleotide that is introduced or present in an increased copy number may be an endogenous gene or an exogenous gene. The endogenous gene refers to a gene that exists in a genetic material included in a microorganism. The exogenous gene refers to a gene that is introduced into a host cell, such as a gene that is integrated into a host cell genome, wherein the introduced gene may be homologous or heterologous with respect to the host cell genome

The expression “increased copy number” may include a copy number increase by an introduction or amplification of the gene. The expression “increased copy number” may also include a copy number increase by genetically manipulating a cell that does not have a gene so as to have the gene in the cell. The introduction of the gene may occur by using a vehicle such as a vector. The introduction may be a transient introduction, in which the gene is not integrated into the genome, or integration into the genome. The introduction may, for example, occur by introducing a vector inserted with a polynucleotide encoding a desired polypeptide into the cell and then replicating the vector in the cell or integrating the polynucleotide into the genome of the cell and then replicating the polynucleotide together with the replication of the genome.

As used herein, the term “gene” refers to a nucleic acid segment expressing a specific protein, and the gene may or may not include one or more regulatory sequences which are nucleic acid segment next to 5′ or 3′ of the coding sequence (e.g., a 5′-non coding sequence and a 3′-non coding sequence).

As used herein, the term “inactivation” may refer to generating a gene that is not expressed at all or a gene that has no activity even when it is expressed (e.g., a gene that produces a non-functional or only partly functional gene product). The term “depression” as used to describe gene expression may refer to a gene whose expression level is reduced compared to a parent yeast cell, or a gene that encodes a protein with decreased activity although it is expressed. The inactivation or depression may be due to mutation, substitution, or deletion of a part or all of a gene, or insertion of at least one base group to a gene. The inactivation or depression may be achieved by gene manipulation such as homogenous recombination, mutation generation, or molecule evolution. When a cell includes a plurality of the same genes or at least two different polypeptide paralogous genes, one or more genes may be inactivated or depressed. The inactivation or depression may be performed by transforming a cell with a vector including some sequences of the gene to a cell, and allowing the sequences to homogeneously recombined with an endogenous gene by culturing the cell, and then by selecting the homogenously recombined cell by using a selection marker.

An increase in an enzyme activity refers to an increase in an expression level, such as an overexpression of a gene encoding an enzyme having the activity, or an increase in the activity of the enzyme itself compared to a cell not having a specific genetic modification resulting in a genetically engineered cell.

As used herein, the term “sequence identity” of a nucleic acid or a polypeptide refers to a degree of similarity (e.g., homology) of base groups or amino acid residues between two aligned sequences, when the two sequences are aligned to match each other as possible, at corresponding positions. The sequence identity is a value that is measured by aligning to an optimum state and comparing the two sequences at a particular comparing region, wherein a part of the sequence within the particular comparing region may be added or deleted compared to a reference sequence. A sequence identity percentage may be calculated, for example, by 1) comparing the two sequences aligned within the whole comparing region to an optimum 2) obtaining the number of matched locations by determining the number of locations represented by the same amino acids of nucleic acids in both of the sequences, 3) dividing the number of the matched locations by the total number of the locations within the comparing region (i.e., a range size), and 4) obtaining a percentage of the sequence identity by multiplying 100 to the result. The sequence identity percent may be determined by using a common sequence comparing program, for example, BLASTN(NCBI), CLC Main Workbench (CLC bio), MegAlign™ (DNASTAR Inc).

In confirming many different polypeptides or polynucleotides having the same or similar function or activity, sequence identities at several levels may be used. For example, the sequence identities may include about 50% or greater, about 55% or greater, about 60% or greater, about 65% or greater, about 70% or greater, about 75% or greater, about 80% or greater, about 85% or greater, about 90% or greater, about 95% or greater, about 96% or greater, about 97% or greater, about 98% or greater, about 99% or greater, or 100%.

The yeast cell may be ascomycota. The ascomycota may be saccharomycetacease. The saccharomycetaceae may be Saccharomyces genus, Kluyveromyces genus, Candida genus, Pichia genus, Issatchenkia genus, Debaryomyces genus, Zygosaccharomyces genus, Shizosaccharomyces genus, or Saccharomycopsis genus. The Saccharomyces genus may be, for example, S. cerevisiae, S. bayanus, S. boulardii, S. bulderi, S. cariocanus, S. cariocus, S. chevalieri, S. dairenensis, S. ellipsoideus, S. eubayanus, S. exiguus, S. florentinus, S. kluyveri, S. martiniae, S. monacensis, S. norbensis, S. paradoxus, S. pastorianus, S. spencerorum, S. turicensis, S. unisporus, S. uvarum, or S. zonatus. The Kluyveromyces genus may be Kluyveromyces lactis, Kluyveromyces marxianus or Kluyveromyces thermotolerans. The Candida genus may be Candida glabrata, Candida boidinii, Candida magnolia, Candida methanosorbosa, Candida sonorensis, or Candida utilis. The Pichia genus may be Pichia stipitis. The Issatchenkia genus may be Issatchenkia orientalis. The Debaryomyces genus may be Debaryomyces hansenii. The Zygosaccharomyces genus may be Zygosaccharomyces bailli or Zygosaccharomyces rouxii. The Shizosaccharomyces genus may be S. cryophilus, S. japonicus, S. octosporus, or S. pombe.

The yeast cell may have a lactate-producing ability. In particular, gene encoding glycerol-3-phosphate dehydrogenase is sufficiently inactivated or depressed to allow the yeast to produce lactate. The activity of glycerol-3-phosphate dehydrogenase may be about 50% or more, about 55% or more, about 60% or more, about 70% or more, about 75% or more, about 80% or more, about 85% or more, about 90% or more, about 95% or more, or about 100% or more reduced compared to an activity of an appropriate control group. The activity of converting glyceraldehydes-3-phosphate to 1,3-diphosphoglycerate may be increased sufficiently enough to produce lactate. The activity may be about 5% or more, about 10% or more, about 15% or more, about 20% or more, about 30% or more, about 50% or more, about 60% or more, about 70% or more, or about 100% or more increased compared to an activity of a control group. Activity of a cell, polypeptide, or enzyme may be inactivated or depressed due to deletion or disruption of a gene encoding the polypeptide or enzyme. As used herein, the “deletion” or “disruption” of the gene includes mutation or deletion of the gene or a regulatory region of the gene (e.g., operator, promoter or terminator regions of the gene), or a part thereof, sufficient to disrupt or delete gene function or the expression of a functional gene product. Mutations include substitutions, insertions, and deletions of one or more bases in the gene or its regulator regions. As a result, the gene is not expressed or has a reduced amount of expression, or the activity of the encoded protein or enzyme is reduced or eliminated. The deletion or disruption of the gene may be accomplished by any suitable genetic engineering technique, such as homologous recombination, mutagenesis, or molecular evolution. When a cell includes a plurality of copies of the same gene or at least two different polypeptide paralogs, at least one gene may be deleted or disrupted.

The glycerol-3-phosphate dehydrogenase (GPD) may be a mitochondrial glycerol-3-phosphate dehydrogenase (GPD2), a cytosolic glycerol-3-phosphate dehydrogenase (GPD1), or a combination thereof.

The mitochondrial glycerol-3-phosphate dehydrogenase (GPD2) may be an enzyme that catalyzes irreversible reduction of dihydroxyacetone phosphate (DHAP) to glycerol-3-phosphate using oxidation FADH₂ to FAD. The GPD2 may belong to EC 1.1.5.3. The GPD2 may include an amino acid sequence having about 50% or more, about 70% or more, about 80% or more, about 90% or more, about 95% or more, about 96% or more, about 97% or more, about 98% or more, about 99% or more, or about 100% or more sequence identity with an amino acid sequence SEQ ID NO: 1. A gene encoding the GPD2 may have a nucleotide sequence of SEQ ID NO: 2.

The cytosolic glycerol-3-phosphate dehydrogenase (GPD1) may be an enzyme catalyzing reduction of dihydroxyacetone phosphate (DHAP) to glycerol-3-phosphate by using oxidation of NAD(P)H to NAD(P)⁺. The GPD1 may be an NAD(P)⁺-dependent enzyme. The GPD1 may belong to EC 1.1.1.8. The GPD1 may be an amino acid sequence having about 50% or more, about 70% or more, about 80% or more, about 90% or more, about 95% or more, about 96% or more, about 97% or more, about 98% or more, about 99% or more, or about 100% or more sequence identity with an amino acid sequence SEQ ID NO: 3. A gene encoding the GPD1 may have a nucleotide sequence of SEQ ID NO: 4.

In the yeast cell, the increased activity of converting glyceraldehyde-3-phosphate to 1,3-diphosphoglycerate may be caused by an increased expression of a glyceraldehyde-3 phosphate dehydrogenase.

The increase in the expression may be caused by an increase in the copy number of the gene or mutation of a regulation region of the gene. The increased copy number of the gene may be due to amplification of an endogenous gene or introduction of an exogenous gene. The mutation of the regulation region of the gene may be due to mutation of a regulation region of an endogenous gene. The exogenous gene may be a homogenous or heterogenous gene.

The polypeptide having an activity of converting glyceraldehyde-3-phosphate to 1,3-diphosphoglycerate may be an enzyme catalyzing conversion of glyceraldehyde-3-phosphate to 1,3-diphosphoglycerate by using reduction of NAD(P)⁺ to NAD(P)H. The enzyme may belong to EC 1.2.1.12. The enzyme may be TDH1. The TDH1 may be a minor isoform of glyceraldehyde-3-phosphate dehydrogenase. When a cell enters a stationary phase, the TDH1 is synthesized and thus may not be expressed under normal conditions. Expression of the TDH1 may increase under cytosolic redox imbalance causing reductive stress. The reductive stress may be NADH-reductive stress. The TDH1 may be related to defense mechanisms of a cell. The enzyme may include an amino acid sequence having about 50% or more, about 70% or more, about 80% or more, about 90% or more, about 95% or more, about 96% or more, about 97% or more, about 98% or more, about 99% or more, or about 100% sequence identity with an amino acid sequence of SEQ ID NO: 5. A gene encoding the enzyme may have a nucleotide sequence of SEQ ID NO: 6.

In the yeast cell, an activity of converting glyceraldehyde-3-phosphate to 1,3-diphosphoglycerate may indicate introduction of a gene encoding a polypeptide that converts glyceraldehyde-3-phosphate to 1,3-diphosphoglycerate. The gene encoding a polypeptide converting glyceraldehyde-3-phosphate to 1,3-diphosphoglycerate may have a nucleotide sequence of SEQ ID NO: 6.

In the yeast cell, an activity of converting pyruvate to acetaldehyde, an activity of converting lactate to pyruvate, or a combination thereof may be further removed or depressed. The term “depressed” may refer to an activity of the genetically engineered yeast cell compared to that of a parent yeast cell.

The yeast cell may have an inactivated or depressed gene encoding a polypeptide that converts pyruvate to acetaldehyde. The polypeptide that converts pyruvate to acetaldehyde may be an enzyme that belongs to EC 4.1.1.1. For example, the polypeptide is a pyruvate decarboxylase. The polypeptide that converts pyruvate to acetaldehyde may include an amino acid sequence having about 50% or more, about 70% or more, about 80% or more, about 90% or more, about 95% or more, about 96% or more, about 97% or more, about 98% or more, about 99% or more, or about 100% or more sequence identity with an amino acid sequence of SEQ ID NO: 7. The gene encoding the polypeptide that converts pyruvate to acetaldehyde may have a nucleotide sequence of SEQ ID NO: 8. The gene may be pdc1 encoding pyruvate decarboxylase (PDC).

In the yeast cell, the gene encoding the polypeptide that converts lactate to pyruvate may be inactivated or depressed. The polypeptide that converts lactate to pyruvate may be a cytochrome c-dependent enzyme. The polypeptide that converts lactate to pyruvate may be a lactate cytochrome-c oxydoreductase (CYB2). The lactate cytochrome c-oxydoreductase may be an enzyme that belongs to EC 1.1.2.4 acting on D-lactate or EC 1.1.2.3 acting on L-lactate. The polypeptide that converts lactate to pyruvate may include an amino acid sequence having about 50% or more, about 70% or more, about 80% or more, about 90% or more, about 95% or more, about 96% or more, about 97% or more, about 98% or more, about 99% or more, or about 100% or more sequence identity with an amino acid sequence of SEQ ID NO: 9. The polypeptide that converts lactate to pyruvate may have a nucleotide sequence of SEQ ID NO: 10.

In the yeast cell, an activity of converting pyruvate to lactate may increase. The activity of converting pyruvate to lactate may increase due to an increase in expression of a polypeptide converting pyruvate to lactate. The increase in expression is same as described above.

The polypeptide that converts pyruvate to lactate may be a lactate dehydrogenase. The lactate dehydrogenase may catalyze conversion of pyruvate to lactate. The lactate dehydrogenase may be a NAD(P)-dependent enzyme, acting on L-lactate or D-lactate. The NAD(P)-dependent enzyme may be an enzyme that belongs to EC 1.1.1.27 acting on L-lactate or EC 1.1.1.28 acting on D-lactate. The lactate dehydrogenase may have an amino acid sequence having about 50% or more, about 70% or more, about 80% or more, about 90% or more, about 95% or more, about 96% or more, about 97% or more, about 98% or more, about 99% or more, or about 100% or more sequence identity with an amino acid sequence of SEQ ID NO: 11. A gene encoding the lactate dehydrogenase may have a nucleotide sequence of SEQ ID NO:

A polynucleotide encoding a lactate dehydrogenase (also may be referred to as “LDH”) may be included in a genome of a yeast cell. When a polynucleotide encoding LDH functions for production of active proteins in a cell, the polynucleotide is considered “functional” in a cell. A polynucleotide encoding LDH is specific in production of L-LDH or D-LDH, and thus a yeast cell including the polynucleotide encoding LDH may produce a L-lactate enantiomer, a D-lactate enantiomer, or a salt thereof.

The yeast cell may include a polynucleotide that encodes one lactate dehydrogenase or multiple polynucleotides that encodes 1 to 10 copies of lactate dehydrogenase. The multiple polynucleotides may encode, for example, 1 to 8, 1 to 5, 1 to 4, or 1 to 3 copies of lactate dehydrogenase. When the yeast cell includes the polynucleotides encoding multiple copies of lactate dehydrogenase, each of the polynucleotides may be a copy of the same polynucleotide or may include a copy of a polynucleotide that encodes at least two different lactate dehydrogenases. Multiple copies of a polynucleotide encoding exogenous lactate dehydrogenase may be included in the same locus or in multiple loci within a host cell's genome.

The polynucleotide encoding LDH may be derived from bacteria, yeast, fungi, mammals, or reptiles. The polynucleotide may encode LDH of at least one selected from Pelodiscus sinensis japonicus, Ornithorhynchus anatinus, Tursiops truncatus, and Rattus norvegicus.

The yeast cell has a glycerol-3-phosphate dehydrogenase converting dihydroxyacetone phosphate to glycerol-3-phosphate is inactivated or depressed and an increased activity of converting glyceraldehyde-3-phosphate to 1,3-diphosphoglycerate, wherein, in the yeast cell, a gene encoding glyceraldehyde-3-phosphate dehydrogenase or a polypeptide that convert glyceraldehyde-3-phosphateis to 1,3-diphosphoglycerate is introduced; and a gene encoding a polypeptide that converts pyruvate to acetaldehyde, a gene encoding a polypeptide that converts lactate to pyruvate, or a combination thereof may be inactivated or depressed, and the yeast cell is derived from Saccharomyces cerevisiae.

Also provided is a method of preparing a genetically modified yeast cell, the method comprising introducing into a yeast cell an exogenous gene encoding glyceraldehyde-3-phosphate dehydrogenase; partially or totally inactivating in the yeast cell a gene encoding a polypeptide that converts pyruvate to acetaldehyde, a gene encoding a polypeptide that converts lactate to pyruvate, or a combination thereof; and introducing into the yeast cell an exogenous gene encoding a polypeptide that converts pyruvate to lactate. All aspects of the yeast cell, exogenous genes introduced therein, and genes inactivated therein are as described with respect to the yeast cell itself.

According to another embodiment of the present invention, a method of producing lactate is provided, wherein the method includes culturing the yeast cell described above in a cell culture medium, whereby the yeast cell produces lactate; and collecting lactate from the culture.

The culturing may be performed in a carbon source, for example, a medium containing glucose. The medium used in the culturing of a yeast cell may be a common medium suitable for growth of a host cell such as a minimal or composite medium containing appropriate supplements. A suitable medium may be purchased from commercial suppliers or may be prepared according to a known preparation method.

The medium used in the culturing may be a medium that satisfies particular conditions for growing a yeast cell. The medium may be one selected from the group consisting of a carbon source, a nitrogen source, a salt, trace elements, and a combination thereof. A pH of a fermented solution may be controlled to be maintained in a range of about 2 to about 7.

The culturing of the yeast cell may be a continuous type, a semi-continuous type, a batch type, or a combination thereof.

The culturing condition for obtaining lactate from the genetically engineered yeast cell may be appropriately controlled. The culturing may be performed in an aerobic or anaerobic condition. For example, the yeast cell is cultured under an aerobic condition for its proliferation, and then, the yeast cell is cultured under an anaerobic condition to produce lactate. The anaerobic condition may include a dissolved oxygen (DO) concentration of 0% to 10%, for example, 0% to 8%, 0% to 6%, 0% to 4%, or 0% to 2%.

The term “culture condition” indicates a condition for culturing a yeast cell. Such culture condition may be, for example, a carbon source, a nitrogen source, or an oxygen condition for the yeast cell to use. The carbon source used by the yeast cell includes monosaccharides, disaccharides, or polysaccharides. In particular, the carbon source may be glucose, fructose, mannose, or galactose. The nitrogen source used by the yeast cell may include an organic nitrogen compound or an inorganic nitrogen compound. In particular, the nitrogen source may be an amino acid, amide, amine, a nitrate, or an ammonium salt. The oxygen condition for culturing the yeast cell includes an aerobic condition of a normal oxygen partial pressure, a low-oxygen condition including 0.1% to 10% of oxygen in the atmosphere, or an anaerobic condition without oxygen. A metabolic pathway may be modified in accordance with the carbon source or the nitrogen source that may be practically used by the yeast cell.

The obtaining of the lactate from the culture may be performed by separating the lactate from the culture by using a method commonly known in the art. The separation method may be centrifuge, filtration, ion-exchange chromatography, or crystallization. For example, the culture may be centrifuged at a low rate to remove a biomass, and the supernatant resulting therefrom may be separated through ion-exchange chromatography.

The present invention will be described in further detail with reference to the following examples. These examples are for illustrative purposes only and are not intended to limit the scope of the present invention.

### Example 1

### Preparation of TDH1 Overexpression Vector

A cassette for overexpressing TDH1 that encodes one of glyceraldehydes-3-phosphate dehydrogenases (Tdh) was prepared in the manner as follows. First, PCR was performed by using a genomic DNA of S. cerevisiae CEN.PK2-1D as a template and primers of SEQ ID NOS: 13 and 14. The PCR condition was as follows: 4 minutes at 95° C., 30 seconds of denaturation at 94° C., 30 seconds at 52° C., 1 minutes of an extension cycle at 72° C. repeated 30 times, and 10 minutes at 72° C. The PCR product thus obtained was digested with SacI and XbaI, and the resultant was introduced to p416-GPD (ATCC® 87360™), producing p416-PDCp.

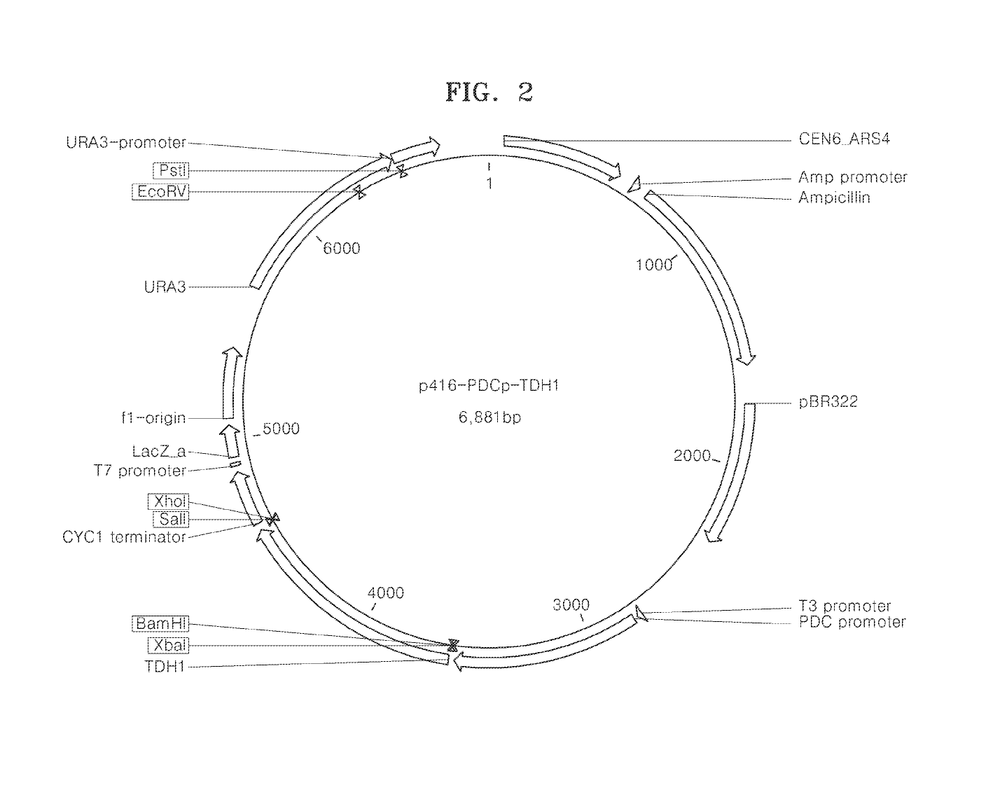

Then, PCR was performed by using a genomic DNA of S. cerevisiae as a template and primers of SEQ ID NOS: 15 and 16. The PCR condition was as follows: 4 minutes at 95° C., 30 seconds of denaturation at 94° C., 30 seconds at 52° C., 3 minutes of an extension cycle at 72° C. repeated 30 times, and 10 minutes at 72° C. The PCR product thus obtained and the prepared p416-PDCp were digested with BamHI and EcoRI and ligated, producing p416-PDCp-TDH1. FIG. 2 is a schematic view of an overexpression vector for overexpressing TDH1. As shown in FIG. 2, p416-PDCp-TDH1 includes TDH1 that would be expressed under control of a PDC promoter.

### Example 2

### Preparation of TDH1 Gene Overexpression and GPD Gene Deletion Vectors

In order to delete GPD2 encoding one of glycerol-3-phosphate dehydrogenases (Gpd) by using a homogenous recombination method, a gene exchange vector was prepared in the manner as follows to.

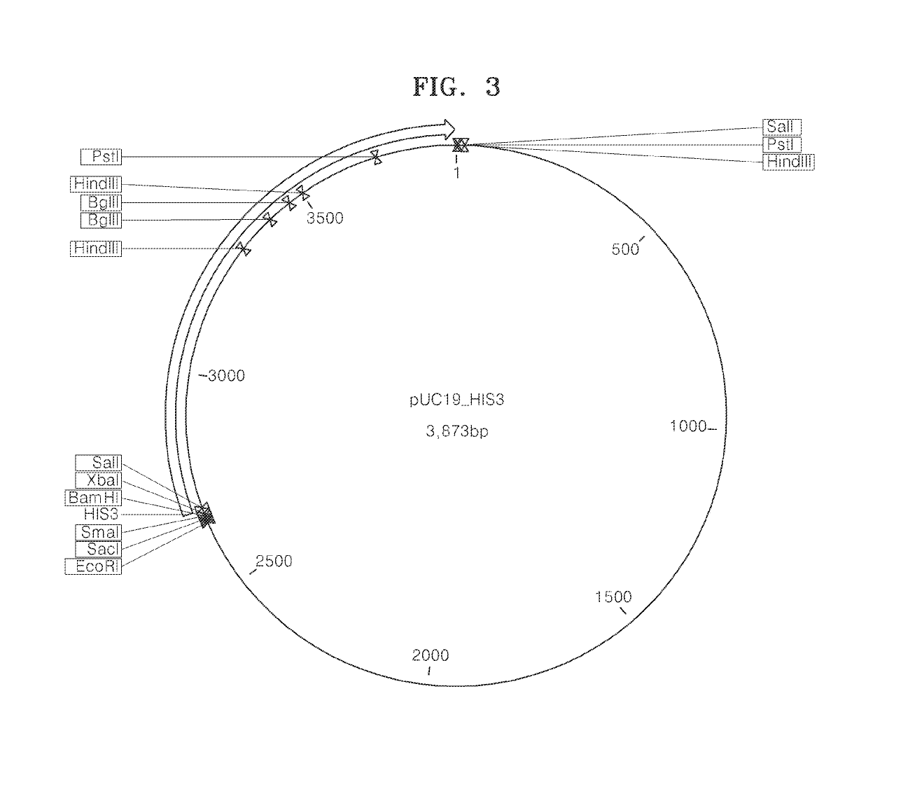

A his3 gene was cloned by using a pUC19 (New England Biolabs Inc.) vector as a template and primers of SEQ ID NOS: 17 and 18. The resulting PCR fragment and pUC19 vector was digested with SalI and ligated, producing a pUC19-HIS3 vector. FIG. 3 is a schematic view illustrating the pUC19-HIS3 vector, in which histidine3-gene, i.e., an auxotrophic marker, was inserted, and the pUC19-HIS3 vector was a mother vector for deleting GPD2, which will be described later, and for preparing a cassette to overexpress TDH1.

Then, PCR was performed by using p416-PDCp-TDH1 prepared in Example 1 as a template and primers of SEQ ID NOS: 19 and 20. The PCR product thus obtained and the pUC19-HIS3 vector were digested with SacI and ligated to prepare a pUC19-PDCp-TDH1-HIS3 vector.

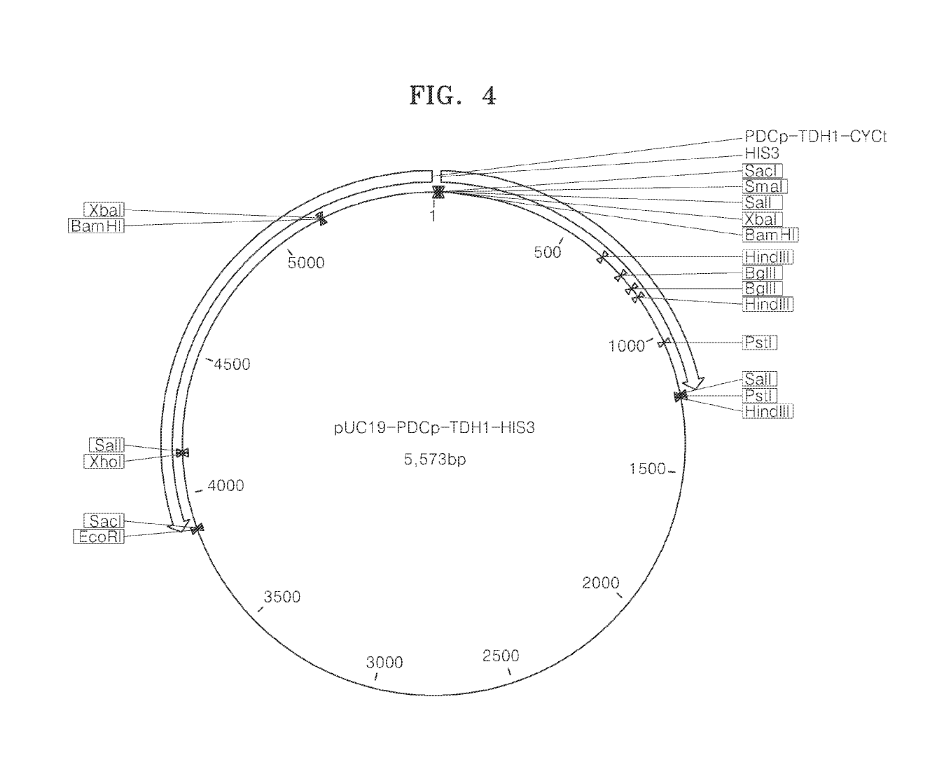

FIG. 4 is a schematic view illustrating a pUC19-PDCp-TDH1-HIS3 vector. The pUC19-PDCp-TDH1-HIS3 vector is a template for preparing a cassette for deleting GPD2 and overexpressing TDH1 by having the pUC19-HIS3 shown in FIG. 3 as a mother vector. Then, PCR was performed by using the prepared pUC19-PDCp-TDH1-HIS3 vector as a template and primers of SEQ ID NOS: 21 and 22 to delete GPD2, and thus a cassette inserted with TDH1 was prepared. The PCR condition was as follows: 4 minutes at 95° C., 30 seconds of denaturation at 94° C., 30 seconds at 52° C., 3 minutes of an extension cycle at 72° C. repeated 30 times, and 10 minutes at 72° C.

Also, in order to prepare a control group strain, a cassette for overexpressing TDH1 was prepared by using the prepared pUC19-PDCp-TDH1-HIS3 vector as a template. The cassette for overexpressing TDH1 from the prepared pUC19-PDCp-TDH1-HIS3 vector, a process was performed as follows. PCR was performed on the prepared pUC19-PDCp-TDH1-HIS3 vector by using primers of SEQ ID NO: 23 and 24 to prepare a cassette introduced with TDH1 at a position of trp1. The PCR condition was as follows: 4 minutes at 95° C., 30 seconds of denaturation at 94° C., 30 seconds at 52° C., 3 minutes of an extension cycle at 72° C. repeated 30 times, and 10 minutes at 72° C.

### Example 3

### Preparation of KCTC12415BP Δ GPD2+TDH1 Strain and KCTC12415BP+TDH1 Strain

A mutant strain (KCTC12415BP Δ GPD2+TDH1) introduced with TDH1 and a mutant strain (KCTC12415BP+TDH1) introduced with TDH1 at the same time when GPD2 was deleted from Saccharomyces cerevisiae were prepared.

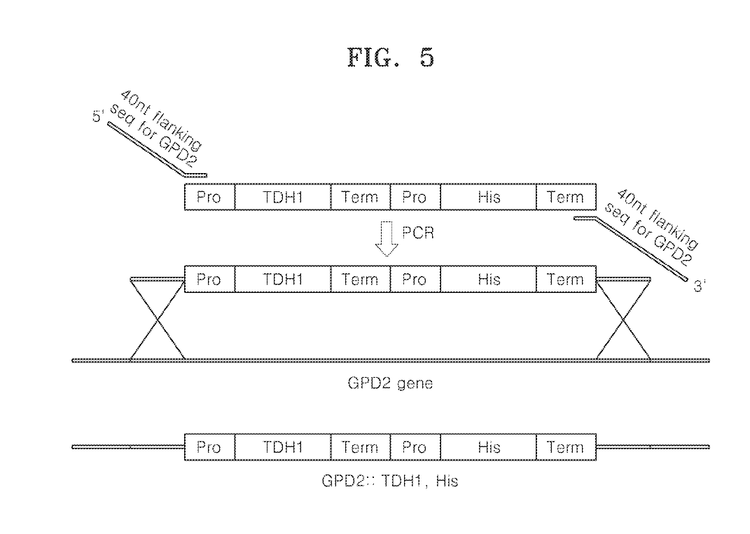

In order to prepare KCTC12415BP Δ GPD2+TDH1 strain, a process was performed as follows. FIG. 5 shows a process of preparing a KCTC12415BP Δ GPD2+TDH1 strain by deleting GPD2 from a mother strain KCTC12415BP. KCTC12415BP (pdc1Δ::LDH cyb2Δ::LDH gpd1Δ::LDH) was spread on a YPD plate (including 10 g of yeast extract, 20 g of peptone, and 20 g of glucose) and incubated for 24 hours at 30° C., and then, a colony obtained therefrom was inoculated in about 10 ml of a YPD liquid medium and cultured for about 18 hours at 30° C. The sufficiently grown culture solution was inoculated in about 50 ml of a YPD liquid medium contained in a 250 ml-flask at a concentration of 1% (v/v) and incubated in an incubator at a rate of about 230 rpm and at 30° C.

After about 4 to 5 hours, when the OD₆₀₀ reached about 0.5, the culture was centrifuged at a rate of about 4,500 rpm for about 10 minutes to harvest cells, and the cells were resuspended in a lithium acetate solution at a concentration of about 100 mM. Then, the cells were harvested by performing centrifugation at a rate of about 4,500 rpm for about 10 minutes, resuspended in a lithium acetate solution at a concentration of about 1 M including about 15% of glycerol, and then divided into a volume of about 100 μl each.

In order to express TDH1 at the same time deleting GPD2, a cassette, from which the GPD2 prepared in Example 2 was deleted and TDH1 was inserted therein, was mixed with 50% of polyethylene glycol and a single stranded carrier DNA and reacted in a water tub for about 1 hour at 42° C., and then, the culture solution was spread on a histidine-free minimal agar plate (including YSD, 6.7 g/L yeast nitrogen base without amino acids, 1.4 g/L Amino acid dropout mix (-his)) and grown for about 24 hours or more at 30° C. Eight colonies (mutant strains) grown on the plate were selected, patched onto the fresh YSD (-his) minimal agar plate, and at the same time, inoculated into a YSD (-his) liquid medium to isolate the genomic DNA from the above mutant strains by using a commonly used kit (Gentra Puregene Cell kit, Qiagen, USA). In order to confirm deletion of GPD2 by using the genomic DNA of the isolated mutant strain as a template, PCR was performed by using primers of SEQ ID NOS: 25 and 26, and then, electrophoresis was performed on the obtained PCR product to confirm deletion of GPD and insertion of the TDH expression cassette. As a result, Saccharomyces cerevisiae CEN.PK2-1D (pdc1Δ::LDH cyb2Δ::LDH gpd1Δ::LDH gpd2 Δ::TDH1) was obtained, and the strain thus obtained was named KCTC12415BP ΔGPD2+TDH1.

Also, a process for preparing KCTC12415BP+TDH1 strain was performed as follows. KCTC12415BP (pdc1Δ::LDH cyb2Δ::LDH gpd1Δ::LDH) was spread on a YPD plate (including 10 g of yeast extract, 20 g of peptone, and 20 g of glucose) and incubated for 24 hours at 30° C., and then, a colony obtained therefrom was inoculated in about 10 ml of a YPD liquid medium and cultured for about 18 hours at 30° C. The sufficiently grown culture solution was inoculated in about 50 ml of a YPD liquid medium contained in a 250 ml-flask at a concentration of 1% (v/v) and incubated in an incubator at a rate of about 230 rpm and at 30° C.

After about 4 to 5 hours, when the OD₆₀₀ reached about 0.5, the culture was centrifuged at a rate of about 4,500 rpm for about 10 minutes to harvest cells, and the cells were resuspended in a lithium acetate solution at a concentration of about 100 mM. Then, the cells were harvested by performing centrifugation at a rate of about 4,500 rpm for about 10 minutes, resuspended in a lithium acetate solution at a concentration of about 1 M including about 15% of glycerol, and then divided into a volume of about 100 μl each.

In order to express TDH1, a cassette prepared for inserting TDH1 at a position of trp1 prepared in Example 1 was mixed with 50% of polyethylene glycol and a single stranded carrier DNA and reacted in a water tub for about 1 hour at 42° C., and then, the culture solution was spread on a histidine-free minimal agar plate (including YSD, 6.7 g/L yeast nitrogen base without amino acids, 1.4 g/L Amino acid dropout mix (-his)) and grown for about 24 hours or more at 30° C. Eight colonies (mutant strains) grown on the plate were selected, patched onto the fresh YSD (-his) minimal agar plate, and at the same time, inoculated into a YSD (-his) liquid medium to isolate the genomic DNA from the above mutant strains by using a commonly used kit (Gentra Puregene Cell kit, Qiagen, USA). In order to confirm deletion of TPR1 by using the genomic DNA of the isolated mutant strain as a template, PCR was performed by using primers of SEQ ID NOS: 27 and 28, and then, electrophoresis was performed on the obtained PCR product to confirm insertion of the TDH1 expression cassette. As a result, Saccharomyces cerevisiae CEN.PK2-1D (pdc1Δ::LDH cyb2Δ::LDH gpd1Δ::LDH trp1Δ::TDH1) was obtained.

Table 1 summarizes genotypes of the prepared KCTC12415BPΔGPD2+TDH1 and KCTC12415BP+TDH1 strains and KCTC12415BP, which is a starting strain. A genotype of KCTC12415BP, i.e., a starting strain is CEN.PK2-1D (MATα ura3-52; trp1-289; leu2-3, 112; his3 Δ 1; MAL2-8^{c}; SUC2, EUROSCARF accession number: 30000B).

TABLE 1

| Strain | Genotype |
|---|---|
| CEN.PK2-1D | MATα ura3-52; trp1-289; leu2-3, 112; |
| | his3 Δ 1; MAL2-8^{C}; SUC2 |
| KCTC12415BP | CEN.PK2-1D, pdc1Δ::LDH |
| | cyb2Δ::LDH gpd1Δ::LDH |
| KCTC12415BP+TDH1 | CEN.PK2-1D, pdc1Δ::LDH |
| | cyb2Δ::LDH gpd1Δ::LDH trp1 |
| | Δ::TDH1 |
| KCTC12415BPΔGPD2+TDH1 | CEN.PK2-1D, pdc1Δ::LDH |
| | cyb2Δ::LDH gpd1Δ::LDH gpd2 |
| | Δ::TDH1 |

### Example 4

### Production of Pure L-Lactate Using KCTC12415BPΔGPD2+TDH1 Strain

The KCTC12415BPΔGPD2+TDH1 strain prepared in Example 3 was spread on a YPD agar plate and grown for about 24 hours or more at 30° C., inoculated into 100 ml of YPD including 80 g/L of glucose, and incubated in an anaerobic condition for about 16 hours or more at 30° C. Fermentation was performed by separately inoculating 100 ml of the culture of the KCTC12415BPΔGPD2+TDH1 strain into a bioreactor containing 1 L of a synthesis medium, and the fermentation condition included initially 60 g/L of glucose and 20 g/L of yeast extract at 30° C. During the fermentation, pH was maintained at pH 5 by using 5N Ca(OH)₂ up to 16 hours, pH 4.5 up to 24 hours, and pH 3.0 up to 60 hours, and a concentration of glucose was maintained at 20 g/L. Additional synthesis medium components included 50 g/L of K₂HPO₄, 10 g/L of MgSO₄, 0.1 g/L of tryptophan, and 0.1 g/L of histidine in addition to glucose.

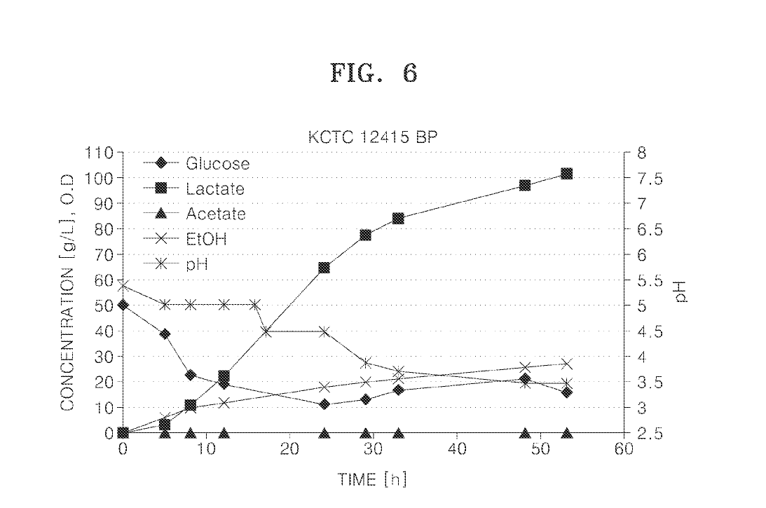

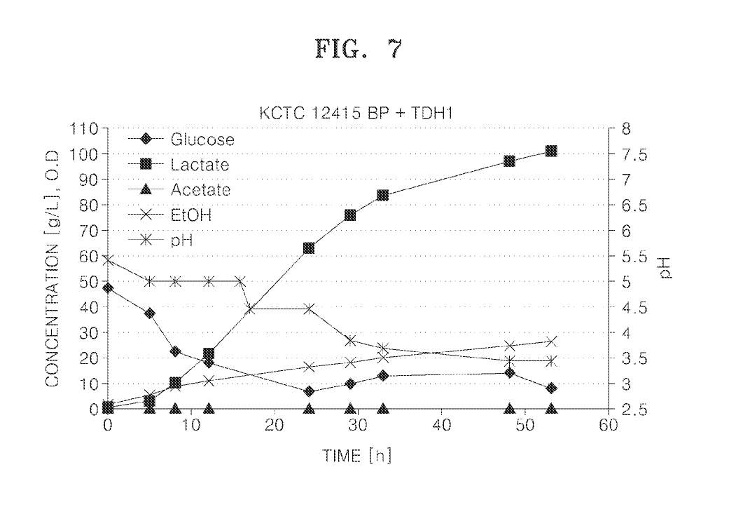

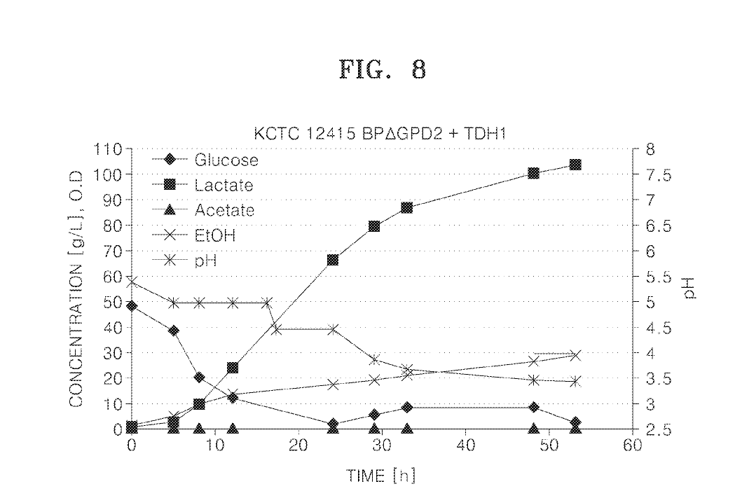

A cell concentration in the culture was estimated by using a spectrophotometer, samples were periodically obtained from the bioreactor during the fermentation, the samples thus obtained were centrifuged at 13,000 rpm for 10 minutes, and then metabolic products and concentrations of lactate and glucose of the supernatants were analyzed by high pressure liquid chromatography (HPLC). FIGS. 6, 7 and 8 illustrates culture characters of the mother strain KCTC12415BP, KCTC12415BP+TDH1 and the KCTC12415BPΔGPD2+TDH1 strain. As shown in FIGS. 6, 7, and 8 and Table 2, the recombined KCTC12415BPΔGPD2+TDH1 strain has an excellent lactate productivity and an increased percent yield compared to that of the mother strain. A lactate productivity of the recombined strain was increased from about 95.9 g/L to about 100.4 g/L compared to that of the control group, KCTC12415BP. Also, a percent yield of the recombined strain was increased from about 53.8% to about 54.1%. The percent yield is a percentage of the produced lactate (g) per the total consumed lactate (g). On the other hand, a lactate productivity and a percent yield of the KCTC12415BP+TDH1 recombined strain were not improved.

TABLE 2

| | Light | | | | |
|---|---|---|---|---|---|
| | ab- | lactate | | EtOH | |
| | sor- | Concen- | | Concen- | |
| | bance | tration | Yield | tration | Yield |
| Strain | (OD) | (g/L) | (%) | (g/L) | (%) |
| KCTC12415BP | 13.45 | 95.9 | 53.80 | 23.5 | 13.20 |
| KCTC12415BP+TDH1 | 13.1 | 96.6 | 53.60 | 23.3 | 12.90 |
| KCTC12415BPΔGPD2+TDH1 | 14.1 | 100.4 | 54.10 | 25.5 | 13.70 |

[Accession Number]

Research Center Name: Korean Collection for Type Cultures (KTCT)

Accession Number: KCTC 12415BP

Accession Date: May 30, 2013

As described above, according to the one or more of the above embodiments of the present invention, a yeast cell may have lactate productivity, and a method of producing lactate may produce lactate efficiently.

It should be understood that the exemplary embodiments described therein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A genetically modified yeast cell comprising increased glyceraldehyde-3-phosphate dehydrogenase activity in converting glyceraldehyde-3-phosphate to 1,3-diphosphoglycerate as compared to a parent yeast cell not having an increased glyceraldehyde-3-phosphate dehydrogenase activity, and a deletion or disruption mutation of a gene encoding a polypeptide that converts dihydroxyacetone phosphate to glycerol-3-phosphate, and glycerol-3-phosphate dehydrogenase activity in converting dihydroxyacetone phosphate to glycerol-3-phosphate is reduced compared to a parent yeast cell not having a deletion or disruption mutation of the gene encoding a polypeptide that converts dihydroxyacetone phosphate to glycerol-3-phosphate.

2. The genetically modified yeast cell of claim 1, wherein the yeast cell is of Saccharomyces genus, Kluyveromyces genus, Candida genus, Pichia genus, Issatchenkia genus, Debaryomyces genus, Zygosaccharomyces genus, Shizosaccharomyces genus, or Saccharomycopsis genus.

3. The genetically modified yeast cell of claim 1, wherein the yeast cell is of Saccharomyces genus.

4. The genetically modified yeast cell of claim 1, wherein the glycerol-3-phosphate dehydrogenase is a mitochondrial glycerol-3-phosphate dehydrogenase (GPD2), a cytosolic glycerol-3-phosphate dehydrogenase (GPD1), or a combination thereof.

5. The genetically modified yeast cell of claim 4, wherein the glycerol-3-phosphate dehydrogenase is a mitochondrial glycerol-3-phosphate dehydrogenase that has about 95% or more sequence identity with the amino acid sequence of SEQ ID NO: 1.

6. The genetically modified yeast cell of claim 4, wherein the glycerol-3-phosphate dehydrogenase is a cytosolic glycerol-3-phosphate dehydrogenase that has about 95% or more sequence identity with the amino acid sequence of SEQ ID NO: 3.

7. The genetically modified yeast cell of claim 4, wherein the glycerol-3-phosphate dehydrogenase is a mitochondrial glycerol-3-phosphate dehydrogenase encoded by a gene that comprises SEQ ID NO: 2.

8. The genetically modified yeast cell of claim 4, wherein the glycerol-3-phosphate dehydrogenase is a cytosolic glycerol-3-phosphate dehydrogenase encoded by a gene that comprises SEQ ID NO: 4.

9. The genetically modified yeast cell of claim 4, wherein the activity of glycerol-3-phosphate dehydrogenase is reduced due to substitution, addition, or deletion of a part of, or all of, a gene encoding the glycerol-3-phosphate dehydrogenase.

10. The genetically modified yeast cell of claim 1, wherein the genetically modified yeast cell comprises the modification of a regulatory sequence for expressing gene encoding glyceraldehyde-3-phosphate dehydrogenase.

11. The genetically modified yeast cell of claim 10, wherein the glyceraldehyde-3-phosphate dehydrogenase is TDH1.

12. The genetically modified yeast cell of claim 1, wherein the genetically modified yeast cell comprises an exogenous gene encoding glyceraldehyde-3-phosphate dehydrogenase.

13. The genetically modified yeast cell of claim 10, wherein the glyceraldehyde-3-phosphate dehydrogenase has an amino acid sequence with about 95% or more sequence identity to SEQ ID NO: 5.

14. The genetically modified yeast cell of claim 10, wherein the genetically modified yeast cell comprises SEQ ID NO: 6.

15. The genetically modified yeast cell of claim 1, wherein the yeast cell has reduced activity of a polypeptide converting pyruvate to acetaldehyde, reduced activity of a polypeptide converting lactate to pyruvate, or a combination thereof, as compared to a parent yeast cell.

16. The genetically modified yeast cell of claim 1, wherein the yeast has increased activity of converting pyruvate to lactate as compared to a parent yeast cell.

17. The genetically modified yeast cell of claim 3, wherein the yeast cell comprises
an exogenous gene encoding glyceraldehyde-3-phosphate dehydrogenase;
a partially or totally inactivated gene encoding a polypeptide that converts pyruvate to acetaldehyde, a partially or totally inactivated gene encoding a polypeptide that converts lactate to pyruvate, or a combination thereof; and
an exogenous gene encoding a polypeptide that converts pyruvate to lactate.

18. A method of preparing a genetically modified yeast cell, the method comprising
introducing into a yeast cell an exogenous gene encoding glyceraldehyde-3-phosphate dehydrogenase;
partially or totally inactivating in the yeast cell a gene encoding a polypeptide that converts pyruvate to acetaldehyde, a gene encoding a polypeptide that converts lactate to pyruvate, or a combination thereof; and
introducing into a yeast cell an exogenous gene encoding a polypeptide that converts pyruvate to lactate.

19. A method of producing lactate, the method comprising:
culturing the genetically modified yeast cell of claim 1 in a cell culture medium, whereby the yeast cell produces lactate; and
collecting lactate from the culture.

20. The method of claim 19, wherein the yeast is cultured under anaerobic conditions.


SEQUENCE LISTING (28 sequences)

SEQ ID NO: 1 - protein, 440 aa, Saccharomyces cerevisiae
MLAVRRLTRYTFLKRTHPVLYTRRAYKILPSRSTFLRRSLLQTQLHSKMTAHTNIKQHKHCHEDHPIRRSDSAVSIVHLKRAPFKVTVIGSGNWGTTIAKVIAENTELHSHIFEPEVRMWVFDEKIGDENLTDIINTRHQNVKYLPNIDLPHNLVADPDLLHSIKGADILVFNIPHQFLPNIVKQLQGHVAPHVRAISCLKGFELGSKGVQLLSSYVTDELGIQCGALSGANLAPEVAKEHWSETTVAYQLPKDYQGDGKDVDHKILKLLFHRPYFHVNVIDDVAGISIAGALKNVVALACGFVEGMGWGNNASAAIQRLGLGEIIKFGRMFFPESKVETYYQESAGVADLITTCSGGRNVKVATYMAKTGKSALEAEKELLNGQSAQGIITCREVHEWLQTCELTQEFPLFEAVYQIVYNNVRMEDLPEMIEELDIDDE

SEQ ID NO: 2 - DNA, 1323 bp, Saccharomyces cerevisiae
atgcttgctgtcagaagattaacaagatacacattccttaagcgaacgcatccggtgttatatactcgtcgtgcatataaaattttgccttcaagatctactttcctaagaagatcattattacaaacacaactgcactcaaagatgactgctcatactaatatcaaacagcacaaacactgtcatgaggaccatcctatcagaagatcggactctgccgtgtcaattgtacatttgaaacgtgcgcccttcaaggttacagtgattggttctggtaactgggggaccaccatcgccaaagtcattgcggaaaacacagaattgcattcccatatcttcgagccagaggtgagaatgtgggtttttgatgaaaagatcggcgacgaaaatctgacggatatcataaatacaagacaccagaacgttaaatatctacccaatattgacctgccccataatctagtggccgatcctgatcttttacactccatcaagggtgctgacatccttgttttcaacatccctcatcaatttttaccaaacatagtcaaacaattgcaaggccacgtggcccctcatgtaagggccatctcgtgtctaaaagggttcgagttgggctccaagggtgtgcaattgctatcctcctatgttactgatgagttaggaatccaatgtggcgcactatctggtgcaaacttggcaccggaagtggccaaggagcattggtccgaaaccaccgtggcttaccaactaccaaaggattatcaaggtgatggcaaggatgtagatcataagattttgaaattgctgttccacagaccttacttccacgtcaatgtcatcgatgatgttgctggtatatccattgccggtgccttgaagaacgtcgtggcacttgcatgtggtttcgtagaaggtatgggatggggtaacaatgcctccgcagccattcaaaggctgggtttaggtgaaattatcaagttcggtagaatgtttttcccagaatccaaagtcgagacctactatcaagaatccgctggtgttgcagatctgatcaccacctgctcaggcggtagaaacgtcaaggttgccacatacatggccaagaccggtaagtcagccttggaagcagaaaaggaattgcttaacggtcaatccgcccaagggataatcacatgcagagaagttcacgagtggctacaaacatgtgagttgacccaagaattcccattattcgaggcagtctaccagatagtctacaacaacgtccgcatggaagacctaccggagatgattgaagagctagacatcgatgacgaatag

SEQ ID NO: 3 - protein, 391 aa, Saccharomyces cerevisiae
MSAAADRLNLTSGHLNAGRKRSSSSVSLKAAEKPFKVTVIGSGNWGTTIAKVVAENCKGYPEVFAPIVQMWVFEEEINGEKLTEIINTRHQNVKYLPGITLPDNLVANPDLIDSVKDVDIIVFNIPHQFLPRICSQLKGHVDSHVRAISCLKGFEVGAKGVQLLSSYITEELGIQCGALSGANIATEVAQEHWSETTVAYHIPKDFRGEGKDVDHKVLKALFHRPYFHVSVIEDVAGISICGALKNVVALGCGFVEGLGWGNNASAAIQRVGLGEIIRFGQMFFPESREETYYQESAGVADLITTCAGGRNVKVARLMATSGKDAWECEKELLNGQSAQGLITCKEVHEWLETCGSVEDFPLFEAVYQIVYNNYPMKNLPDMIEELDLHED

SEQ ID NO: 4 - DNA, 1176 bp, Saccharomyces cerevisiae
atgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattag

SEQ ID NO: 5 - protein, 332 aa, Saccharomyces cerevisiae
MIRIAINGFGRIGRLVLRLALQRKDIEVVAVNDPFISNDYAAYMVKYDSTHGRYKGTVSHDDKHIIIDGVKIATYQERDPANLPWGSLKIDVAVDSTGVFKELDTAQKHIDAGAKKVVITAPSSSAPMFVVGVNHTKYTPDKKIVSNASCTTNCLAPLAKVINDAFGIEEGLMTTVHSMTATQKTVDGPSHKDWRGGRTASGNIIPSSTGAAKAVGKVLPELQGKLTGMAFRVPTVDVSVVDLTVKLEKEATYDQIKKAVKAAAEGPMKGVLGYTEDAVVSSDFLGDTHASIFDASAGIQLSPKFVKLISWYDNEYGYSARVVDLIEYVAKA

SEQ ID NO: 6 - DNA, 999 bp, Saccharomyces cerevisiae
atgatcagaattgctattaacggtttcggtagaatcggtagattggtcttgagattggctttgcaaagaaaagacattgaggttgttgctgtcaacgatccatttatctctaacgattatgctgcttacatggtcaagtacgattctactcatggtagatacaagggtactgtttcccatgacgacaagcacatcatcattgatggtgtcaagatcgctacctaccaagaaagagacccagctaacttgccatggggttctctaaagatcgatgtcgctgttgactccactggtgttttcaaggaattggacaccgctcaaaagcacattgacgctggtgccaagaaggttgtcatcactgctccatcttcttctgctccaatgtttgttgttggtgttaaccacactaaatacactccagacaagaagattgtctccaacgcttcttgtaccaccaactgtttggctccattggccaaggttatcaacgatgctttcggtattgaagaaggtttgatgaccactgttcactccatgaccgccactcaaaagactgttgatggtccatcccacaaggactggagaggtggtagaaccgcttccggtaacattatcccatcctctaccggtgctgctaaggctgtcggtaaggtcttgccagaattgcaaggtaagttgaccggtatggctttcagagtcccaaccgtcgatgtttccgttgttgacttgactgtcaagttggaaaaggaagctacttacgaccaaatcaagaaggctgttaaggctgccgctgaaggtccaatgaagggtgttttgggttacaccgaagatgccgttgtctcctctgatttcttgggtgacactcacgcttccatcttcgatgcctccgctggtatccaattgtctccaaagttcgtcaagttgatttcctggtacgataacgaatacggttactccgccagagttgttgacttgatcgaatatgttgccaaggcttaa

SEQ ID NO: 7 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 8 - DNA, 1692 bp, Saccharomyces cerevisiae
atgtctgaaattactttgggtaaatatttgttcgaaagattaaagcaagtcaacgttaacaccgttttcggtttgccaggtgacttcaacttgtccttgttggacaagatctacgaagttgaaggtatgagatgggctggtaacgccaacgaattgaacgctgcttacgccgctgatggttacgctcgtatcaagggtatgtcttgtatcatcaccaccttcggtgtcggtgaattgtctgctttgaacggtattgccggttcttacgctgaacacgtcggtgttttgcacgttgttggtgtcccatccatctctgctcaagctaagcaattgttgttgcaccacaccttgggtaacggtgacttcactgttttccacagaatgtctgccaacatttctgaaaccactgctatgatcactgacattgctaccgccccagctgaaattgacagatgtatcagaaccacttacgtcacccaaagaccagtctacttaggtttgccagctaacttggtcgacttgaacgtcccagctaagttgttgcaaactccaattgacatgtctttgaagccaaacgatgctgaatccgaaaaggaagtcattgacaccatcttggctttggtcaaggatgctaagaacccagttatcttggctgatgcttgttgttccagacacgacgtcaaggctgaaactaagaagttgattgacttgactcaattcccagctttcgtcaccccaatgggtaagggttccattgacgaacaacacccaagatacggtggtgtttacgtcggtaccttgtccaagccagaagttaaggaagccgttgaatctgctgacttgattttgtctgtcggtgctttgttgtctgatttcaacaccggttctttctcttactcttacaagaccaagaacattgtcgaattccactccgaccacatgaagatcagaaacgccactttcccaggtgtccaaatgaaattcgttttgcaaaagttgttgaccactattgctgacgccgctaagggttacaagccagttgctgtcccagctagaactccagctaacgctgctgtcccagcttctaccccattgaagcaagaatggatgtggaaccaattgggtaacttcttgcaagaaggtgatgttgtcattgctgaaaccggtacctccgctttcggtatcaaccaaaccactttcccaaacaacacctacggtatctctcaagtcttatggggttccattggtttcaccactggtgctaccttgggtgctgctttcgctgctgaagaaattgatccaaagaagagagttatcttattcattggtgacggttctttgcaattgactgttcaagaaatctccaccatgatcagatggggcttgaagccatacttgttcgtcttgaacaacgatggttacaccattgaaaagttgattcacggtccaaaggctcaatacaacgaaattcaaggttgggaccacctatccttgttgccaactttcggtgctaaggactatgaaacccacagagtcgctaccaccggtgaatgggacaagttgacccaagacaagtctttcaacgacaactctaagatcagaatgattgaaatcatgttgccagtcttcgatgctccacaaaacttggttgaacaagctaagttgactgctgctaccaacgctaagcaataa

SEQ ID NO: 9 - protein, 591 aa, Saccharomyces cerevisiae
MLKYKPLLKISKNCEAAILRASKTRLNTIRAYGSTVPKSKSFEQDSRKRTQSWTALRVGAILAATSSVAYLNWHNGQIDNEPKLDMNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPKILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEKAIEILRDEIEMSMRLLGVTSIAELKPDLLDLSTLKARTVGVPNDVLYNEVYEGPTLTEFEDA

SEQ ID NO: 10 - DNA, 1776 bp, Saccharomyces cerevisiae
atgctaaaatacaaacctttactaaaaatctcgaagaactgtgaggctgctatcctcagagcgtctaagactagattgaacacaatccgcgcgtacggttctaccgttccaaaatccaagtcgttcgaacaagactcaagaaaacgcacacagtcatggactgccttgagagtcggtgcaattctagccgctactagttccgtggcgtatctaaactggcataatggccaaatagacaacgagccgaaactggatatgaataaacaaaagatttcgcccgctgaagttgccaagcataacaagcccgatgattgttgggttgtgatcaatggttacgtatacgacttaacgcgattcctaccaaatcatccaggtgggcaggatgttatcaagtttaacgccgggaaagatgtcactgctatttttgaaccactacatgctcctaatgtcatcgataagtatatagctcccgagaaaaaattgggtccccttcaaggatccatgcctcctgaacttgtctgtcctccttatgctcctggtgaaactaaggaagatatcgctagaaaagaacaactaaaatcgctgctacctcctctagataatattattaacctttacgactttgaatacttggcctctcaaactttgactaaacaagcgtgggcctactattcctccggtgctaacgacgaagttactcacagagaaaaccataatgcttatcataggatttttttcaaaccaaagatccttgtagatgtacgcaaagtagacatttcaactgacatgttgggttctcatgtggatgttcccttctacgtgtctgctacagctttgtgtaaactgggaaaccccttagaaggtgaaaaagatgtcgccagaggttgtggccaaggtgtgacaaaagtcccacaaatgatatctactttggcttcatgttcccctgaggaaattattgaagcagcaccctctgataaacaaattcaatggtaccaactatatgttaactctgatagaaagatcactgatgatttggttaaaaatgtagaaaagctgggtgtaaaggcattatttgtcactgtggatgctccaagtttaggtcaaagagaaaaagatatgaagctgaaattttccaatacaaaggctggtccaaaagcgatgaagaaaactaatgtagaagaatctcaaggtgcttcgagagcgttatcaaagtttattgacccctctttgacttggaaagatatagaagagttgaagaaaaagacaaaactacctattgttatcaaaggtgttcaacgtaccgaagatgttatcaaagcagcagaaatcggtgtaagtggggtggttctatccaatcatggtggtagacaattagatttttcaagggctcccattgaagtcctggctgaaaccatgccaatcctggaacaacgtaacttgaaggataagttggaagttttcgtggacggtggtgttcgtcgtggtacagatgtcttgaaagcgttatgtctaggtgctaaaggtgttggtttgggtagaccattcttgtatgcgaactcatgctatggtcgtaatggtgttgaaaaagccattgaaattttaagagatgaaattgaaatgtctatgagactattaggtgttactagcattgcggaattgaagcctgatcttttagatctatcaacactaaaggcaagaacagttggagtaccaaacgacgtgctgtataatgaagtttatgagggacctactttaacagaatttgaggatgcatga

SEQ ID NO: 11 - protein, 338 aa, Pelodiscus sinensis japonicus
MSVKELLIQNVHKEEHSHAHNKITVVGVGAVGMACAISILMKDLADELALVDVIEDKLRGEMLDLQHGSLFLRTPKIVSGKDYSVTAHSKLVIITAGARQQEGESRLNLVQRNVNIFKFIIPNVVKYSPDCMLLVVSNPVDILTYVAWKISGFPKHRVIGSGCNLDSARFRYLMGEKLGIHSLSCHGWIIGEHGDSSVPVWSGVNVAGVSLKALYPDLGTDADKEHWKEVHKQVVDSAYEVIKLKGYTSWAIGLSVADLAETVMKNLRRVHPISTMVKGMYGVSSDVFLSVPCVLGYAGITDVVKMTLKSEEEEKLRKSADTLWGIQKELQFHHHHHH

SEQ ID NO: 12 - DNA, 1017 bp, Pelodiscus sinensis japonicus
atgtcagttaaggaattgttgattcaaaatgttcacaaggaagaacattctcatgcacacaataagatcacagtcgttggagtgggcgctgtgggtatggcttgcgccatctctatattgatgaaggacttggctgacgaattggcattggtggatgttatcgaggacaagttgagaggggaaatgcttgatctacaacacggtagtttgtttttgagaacccctaagatcgtaagtggtaaggactattcagttacagctcactctaagctggtaattataacggctggtgctagacagcaagaaggagagtctagactaaacttggttcaaagaaacgttaacatcttcaagtttattattcctaacgtggttaaatacagtccagattgtatgttgttggttgtttctaacccagtcgatatcttgacctacgttgcctggaagatctccggtttcccaaagcatagagtcatcggttctggttgtaatttggattctgctagattcagatacttgatgggtgagaagcttggcatccatagcttgtcgtgtcacggttggatcattggtgaacatggtgactcgtccgtcccagtctggtccggtgttaacgtggctggtgtctcactcaaggctttgtacccagatttgggcactgatgcagataaagaacactggaaggaagtccacaaacaggtggttgacagcgcttacgaggtcatcaagttgaaaggttacacctcttgggccattggtttgtctgtagcagacttggccgagactgttatgaagaaccttagaagggtgcacccaatttccactatggtcaagggtatgtacggtgtttcctccgacgttttcttgtccgtcccatgtgtcttgggttatgccggtatcaccgatgttgttaagatgaccctaaaatctgaagaagaagagaagctacgtaaatctgcggacactctctggggtattcaaaaggaattgcaattccatcatcatcatcatcattaa

SEQ ID NO: 13 - DNA, 23 bp, Artificial Sequence
  Synthetic (forward primer)
gagctcaacaagctcatgcaaag

SEQ ID NO: 14 - DNA, 21 bp, Artificial Sequence
  Synthetic (reverse primer)
tctagagatttgactgtgtta

SEQ ID NO: 15 - DNA, 41 bp, Artificial Sequence
  Synthetic (forward primer)
cgcggatccaaaacgatgatcagaattgctattaacggttt

SEQ ID NO: 16 - DNA, 28 bp, Artificial Sequence
  Synthetic (reverse primer)
gaattcttaagccttggcaacatattcg

SEQ ID NO: 17 - DNA, 31 bp, Artificial Sequence
  Synthetic (forward primer)
cctcctgagtcgacaattcccgttttaagag

SEQ ID NO: 18 - DNA, 30 bp, Artificial Sequence
  Synthetic (reverse primer)
cgaccgtggtcgacccgtcgagttcaagag

SEQ ID NO: 19 - DNA, 26 bp, Artificial Sequence
  Synthetic (forward primer)
gagctcaattaaccctcactaaaggg

SEQ ID NO: 20 - DNA, 26 bp, Artificial Sequence
  Synthetic (reverse primer)
gagctccaaattaaagccttcgagcg

SEQ ID NO: 21 - DNA, 59 bp, Artificial Sequence
  Synthetic (forward primer)
atgcttgctgtcagaagattaacaagatacacattccttagtgctgcaaggcgattaag

SEQ ID NO: 22 - DNA, 59 bp, Artificial Sequence
  Synthetic (reverse primer)
ctattcgtcatcgatgtctagctcttcaatcatctccggtcggctcgtatgttgtgtgg

SEQ ID NO: 23 - DNA, 69 bp, Artificial Sequence
  Synthetic (forward primer)
tgagcacgtgagtatacgtgattaagcacacaaaggcagcttggagtatggtgctgcaaggcgattaag

SEQ ID NO: 24 - DNA, 67 bp, Artificial Sequence
  Synthetic (reverse primer)
aggcaagtgcacaaacaatacttaaataaatactactcagtaataacccggctcgtatgttgtgtgg

SEQ ID NO: 25 - DNA, 22 bp, Artificial Sequence
  Synthetic (forward primer)
gctcttctctaccctgtcattc

SEQ ID NO: 26 - DNA, 22 bp, Artificial Sequence
  Synthetic (reverse primer)
tagtgtacagggtgtcgtatct

SEQ ID NO: 27 - DNA, 21 bp, Artificial Sequence
  Synthetic (forward primer)
gccaaatgatttagcattatc

SEQ ID NO: 28 - DNA, 21 bp, Artificial Sequence
  Synthetic (reverse primer)
aaaaggagagggccaagaggg